SEQUENCE LISTING (15 sequences)

SEQ ID NO: 1 - DNA, 20 bp, Artificial Sequence
  Synthetic Construct
ctaccagcagatgaaccccg

SEQ ID NO: 2 - DNA, 20 bp, Artificial Sequence
  Synthetic Construct
cgacctgcccaccagtcccc

SEQ ID NO: 3 - DNA, 20 bp, Artificial Sequence
  Synthetic Construct
ccccttgccgtcccaagcaa

SEQ ID NO: 4 - DNA, 20 bp, Artificial Sequence
  Synthetic Construct
gctaacgatctctttgatga

SEQ ID NO: 5 - DNA, 20 bp, Artificial Sequence
  Synthetic Construct
ctgaaacagttgttacccgg

SEQ ID NO: 6 - DNA, 20 bp, Artificial Sequence
  Synthetic Construct
tcaacgacttcgacctgctc

SEQ ID NO: 7 - DNA, 20 bp, Artificial Sequence
  Synthetic Construct
gtgatggtggtggtggtgag

SEQ ID NO: 8 - DNA, 19 bp, Artificial Sequence
  Synthetic Construct
gagcgagggagcacattgg

SEQ ID NO: 9 - DNA, 20 bp, Artificial Sequence
  Synthetic Construct
gcgcacctggaagactacta

SEQ ID NO: 10 - DNA, 22 bp, Artificial Sequence
  Synthetic Construct
tgctcttgtctttcagacttcc

SEQ ID NO: 11 - DNA, 22 bp, Artificial Sequence
  Synthetic Construct
ggaagggacagaagatgacagg

SEQ ID NO: 12 - DNA, 20 bp, Artificial Sequence
  Synthetic Construct
ccaggcctctggctgctgag

SEQ ID NO: 13 - DNA, 21 bp, Artificial Sequence
  Synthetic Construct
cggacaatagccctcaggaag

SEQ ID NO: 14 - DNA, 20 bp, Artificial Sequence
  Synthetic Construct
tgcaggtttgttgtgagggt

SEQ ID NO: 15 - DNA, 20 bp, Artificial Sequence
  Synthetic Construct
gctccctttccggaacttca

(19) United States
(12) Patent Application Publication
(10) Pub. No.: US 2021/0254068 A1
(43) Pub. Date: Aug. 19, 2021

(54) GENOME ENGINEERING PRIMARY MONOCYTES

(71) Applicant: Regents of the University of Minnesota, Minneapolis, MN (US)

(72) Inventors: Branden S. MORIARITY, Minneapolis, MN (US); Kanut LAOHARAWEE, Minneapolis, MN (US); Matthew J. JOHNSON, Minneapolis, MN (US)

(73) Assignee: Regents of the University of Minnesota, Minneapolis, MN (US)

(21) Appl. No.: 17/252,556

(22) PCT Filed: Jun. 19, 2019
(86) PCT No.: PCT/US2019/037986

§ 371 Date: Dec. 15, 2020

Related U.S. Application Data
(60) Provisional application No. 62/687,148, filed on Jun. 19, 2018.

(51) Int. Cl. C12N 15/113; C12N 9/22; C12N 5/0786; C12N 15/86; C12N 15/87

(52) U.S. Cl. CPC C12N 15/113; C12N 9/22; C12N 5/0645; C12N 2330/51; C12N 15/87; C12N 2310/20; C12N 2510/00; C12N 15/86

(57) ABSTRACT

The present disclosure relates generally to methods and tools for engineering a genome of a mammalian monocyte. In particular, the present disclosure relates to mammalian monocytes having at least one altered locus, and reagents for production thereof.

### CROSS-REFERENCE TO RELATED APPLICATIONS

This application claims priority to and benefit of U.S. Provisional Application No. 62/687,148, filed Jun. 19, 2018, the disclosure of which is incorporated herein by reference in its entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 144832000840SEQLIST.TXT, date recorded: Jun. 17, 2019, size: 3 KB).

### TECHNICAL FIELD

The present disclosure relates generally to methods and tools for engineering a genome of a mammalian monocyte. In particular, the present disclosure relates to mammalian monocytes having at least one altered locus, and reagents for production thereof.

### BACKGROUND

Monocytes are innate immune cells derived from myeloid-lineage progenitors of hematopoietic stem cells in the bone marrow (Geissmann et al., Science, 327:656-661, 2010). There are 3 subsets of blood circulating monocytes: CD14++/CD16−, CD14++/CD16+, and CD14+/CD16++(Wong et al., Immunologic Research, 53:41-57, 2012). They circulate in the bone marrow, spleen and blood vessels, and are equipped with adhesion receptors and chemokine receptors that mediate their migration and extravasation to tumor and inflammatory sites, where they are activated to differentiate into macrophages or dendritic cells (DCs) (Auffray et al., Annual Review of Immunology, 27:669-692, 2009; and Italiani and Boraschi, Frontiers in Immunology, 5:514, 2014). Macrophages represent the majority of phagocytic cells and DCs represent robust antigen presenting cells, both of which are involved in regulating the innate and adaptive immune system (Yona et al., Immunity, 38:79-91, 2013). However, monocyte activation and differentiation can have side effects such as inflammation, which may lead to inflammatory diseases (Auffray et al., supra, 2009). In addition, monocytes are known to differentiate into tumor-associated macrophages and promote tumor progression (Sica et al., European Journal of Cancer, 42:717-727, 2006; and Richards et al., Cancer Microenvironment, 6:179-191, 2013).

For decades, cytokines and small molecules have been used to manipulate the biological functions of immune cells, including monocytes, macrophages, and DCs ex vivo. However, this manipulation has been reported to cause systemic cytotoxicity in clinical applications. For instance, immune-related adverse events have been reported in recipients of immune checkpoint antibodies (Naidoo et al., Annals of Oncology, 26:2375-2391, 2015). This is due to global effects of immune checkpoint antibodies on multiple types of immune cells (Naidoo et al., Annals of Oncology, 26:2375-2391, 2015). Thus, what the art needs are methods and reagents for selectively manipulating the genotype and phenotype of monocytes.

### SUMMARY

The present disclosure relates generally to methods and tools for engineering a genome of a mammalian monocyte. In particular, the present disclosure relates to mammalian monocytes having at least one altered locus, and reagents for production thereof.

### BRIEF DESCRIPTION OF THE DRAWINGS

FIGS. 1A-1B show schematics of exemplary strategies for CRISPR/Cas9 and adeno-associated virus (AAV)-mediated genome engineering in monocytes. FIG. 1A shows an exemplary strategy for site-specific insertion of an expression cassette lacking an exogenous promoter into the monocyte genome via homology directed repair. FIG. 1B shows an exemplary strategy for site-specific insertion of an expression cassette containing an exogenous promoter into the monocyte genome via homology directed repair.

### DETAILED DESCRIPTION

The present disclosure relates generally to methods and tools for engineering a genome of a mammalian monocyte. In particular, the present disclosure relates to mammalian monocytes having at least one altered locus, and reagents for production thereof.

In particular, the present disclosure describes various methods for genetic engineering of monocytes, as well as their downstream effectors (macrophages and dendritic cells). This is accomplished by introducing at least one alteration into a target locus of a monocyte genome. The alteration may include one or both of a gene knock out and a gene knock in. Manipulation of monocytes via genetic engineering techniques is expected to reduce or eliminate the use of cytokines and small molecules, and consequently to reduce risk of systemic cytotoxicity in clinical settings.

Precise modulation of primary monocytes has multiple applications in the fields of immunotherapy, autoimmunity and enzymopathy. Modulation of monocytes at the genetic level is an attractive route for therapy due to the permanence of treatment and the low risk of rejection by the patient. One approach for gene editing immune cells is to use Clustered Regularly Interspaced Short Palindromic Repeat (CRISPR) a system which induces a double strand break (DSB) within a gene of interest, thereby resulting in the formation of small insertions or deletions (collectively referred to as ‘indels’) created by semi-random repair via the Non-Homologous End Joining (NHEJ) pathway. Alternatively, precise genome alterations can be achieved by the introduction of a DSB along with co-delivery of a DNA template for repair via homology directed repair (HDR).

### I. Definitions

As used herein and in the appended claims, the singular forms “a,” “an,” and “the” include plural references unless indicated otherwise. For example, “a” monocyte includes one or more monocytes.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments. It is understood that aspects and embodiments described herein as “comprising” include “consisting of” and “consisting essentially of” embodiments.

The term “about” as used herein in reference to a value, encompasses from 90% to 110% of that value (e.g., about 20 ms refers to 18 ms to 22 ms and includes 20 ms).

The term “plurality” as used herein in reference to an object refers to three or more objects. For instance, “a plurality of cells” refers to three or more cells, preferably 3, 4, 5, 6, 7, 8, 9, 10, 100, 1,000, 10,000, 100,000, 1,000,000 or more cells.

As used herein, the term “isolated” refers to an object (e.g., monocyte) that is removed from its natural environment (e.g., separated). “Isolated” objects are at least 50% free, preferably 75% free, more preferably at least 90% free, and most preferably at least 95% (e.g., 95%, 96%, 97%, 98%, or 99%) free from other components with which they are naturally associated

As used herein, the term “nucleic acid” includes single-stranded DNA (ssDNA), double-stranded DNA (dsDNA), single-stranded RNA (ssRNA) and double-stranded RNA (dsRNA), modified oligonucleotides and oligonucleosides, or combinations thereof.

The term “subject” refers to mammals. “Mammals” include, but are not limited to, humans, non-human primates (e.g., monkeys), farm animals, sport animals, rodents (e.g., mice and rats) and pets (e.g., dogs and cats).

As used herein, the terms “guide RNA” and “gRNA” refer to at least one nucleic acid that is capable of directing an endonuclease to cleave dsDNA of a target locus of a genome.

### II. Methods for Genome Engineering Mammalian Monocytes

The present disclosure provides methods for engineering a genome of a CD14+ mammalian monocyte, comprising: a) introducing a clustered regularly interspaced short palindromic repeats (CRISPR) system into the CD14+ mammalian monocyte to produce a transfected monocyte, wherein the CRISPR system comprises a i) at least one guide RNA (gRNA) comprising a sequence that anneals to a target locus of the genome, and ii) an endonuclease or a nucleic acid encoding the endonuclease; and b) culturing the transfected monocyte to produce an engineered monocyte comprising at least one alteration of the target locus.

In some embodiments, the CD14+ mammalian monocyte is a primary cell or a mortal cultured cell. In some embodiments, the primary monocyte is isolated from a blood sample (e.g., PBMCs) obtained from a mammalian subject. In some embodiments, the primary monocyte is a monocyte that is freshly isolated. In other embodiments, the primary monocyte was frozen and subsequently thawed before introduction of the CRISPR system.

At least one guide RNA (gRNA) of the present disclosure includes a nucleic acid sequence that is complementary to a strand of the dsDNA of the target locus adjacent to a protospacer adjacent motif (PAM), and a nucleic acid sequence to facilitate assembly of a ribonucleoprotein complex with the endonuclease. For instance, the term gRNA when used in reference to the Alt-R® CRISPR-Cas9 System (Integrated DNA Technologies) includes two RNA molecules: a crRNA and a tracrRNA. In some embodiments, the gRNA may include one or more chemical modifications that increase its nuclease resistance and/or reduce activation of innate immune responses. A chemically modified gRNA may include one or more of the following modifications: 2′-fluoro (2′-F), 2′-O-methyl (2′-O-Me), S-constrained ethyl (cEt), 2′-O-methyl (M), 2′-O-methyl-3′-phosphorothioate (MS), and 2′-O-methyl-3′-thiophosphonoacetate (MSP). The chemically modified gRNA may include a chemical modification as previously described (see, e.g., Hendel et al, Nature Biotechnology, 33:985-989, 2015; and Randar et al., Proc. Natl. Acad. Sci, USA, 112:E7110-7, 2015).

The endonuclease of the present disclosure is suitable for introducing double-stranded breaks in the target locus DNA in an RNA-guided manner. In some embodiments, the RNA-guided endonuclease is Cas9, whereas in other embodiments, the RNA-guided endonuclease is Cpf1. In some embodiments, the methods involve introduction of a donor nucleic acid into the site of the dsDNA break by homologous recombination.

Additionally, based on the present disclosure methods for engineering NK cells and B lymphocytes as described in WO 2017/214569 and WO 2018/049401 can be adapted to methods for engineering mammalian monocytes.

### Electroporation

Introduction of the CRISPR system and optionally the donor nucleic acid into the CD14+ mammalian monocyte may be accomplished by electroporation. In some embodiments, electroporation of the mammalian monocyte comprises exposing the monocyte to from about 1650 to about 2450 volts for a duration of from 5 to 25 milliseconds for from 1 to 3 energy pulses. In some embodiments, electroporation of the CD14+ mammalian monocyte comprises exposing the monocyte to from 1650 to 1750 volts for a duration of about 20 milliseconds for 2 energy pulses. In some embodiments, electroporation of the CD14+ mammalian monocyte comprises exposing the monocyte to about 1700 volts for a duration of about 20 milliseconds for 2 energy pulses. In some embodiments, electroporation of the mammalian monocyte comprises exposing the monocyte to from 1850 to 2450 volts for a duration of from 5 to 25 milliseconds for from 1 to 3 energy pulses. In some embodiments, electroporation of the CD14+ mammalian monocyte comprises exposing the monocyte to from 2000 to 2300 volts for a duration of from 10 to 20 milliseconds for from 1 to 3 energy pulses. In some embodiments, electroporation of the CD14+ mammalian monocyte comprises exposing the monocyte to about 2150 volts for a duration of from about 10 to about 20 milliseconds for 1 or 2 energy pulses. In some preferred embodiments, electroporation of the CD14+ mammalian monocytes is carried out using the Neon® Transfection System (Invitrogen).

### Viral Transduction

Introduction of the CRISPR system and/or the donor nucleic acid into the CD14+ mammalian monocyte may be accomplished by viral transduction. In some embodiments, the donor nucleic acid contained in a viral vector is introduced into the mammalian monocyte, after the CRISPR system was introduced into the mammalian monocyte by another form of delivery (e.g., electroporation, lipofection, etc.). In some embodiments, the viral vector is an adeno-associated virus (AAV), such as an AAV serotype 6 (AAV6). In some embodiments, a promoter-less splice acceptor AAV6 vector encoding a protein of interest and flanked by homology arms to a CRISPR-targeted site may be used as a donor template upon induction of a double strand break at the target site. In other embodiments, the viral vector is a lentivirus, such as a Baboon endogenous virus glycoprotein-pseudotyped (BaEV) lentivirus.

Additionally, based on the present disclosure methods for engineering T lymphocytes using viral vectors as described in WO 2018/081470 and WO 2018/081476 can be adapted to methods for engineering mammalian monocytes.

### Other Delivery Systems

In further embodiments, introduction of the CRISPR system and/or the donor nucleic acid into the CD14+ mammalian monocyte is accomplished using a chemical-based transfection method or a particle-based transfection method. Examples of chemical-based transfection systems include calcium phosphate-mediated transfection and lipid-mediated transfection (lipofection).

### III. Compositions Comprising Engineered Mammalian Monocytes

The present disclosure further provides a plurality of engineered monocytes produced from CD14+ mammalian monocytes according to the methods of Section II above. The present disclosure also provides compositions comprising a plurality of engineered monocytes and a cell culture medium or sterile isotonic solution. Suitable cell culture media include but are not limited to Dulbecco's modified Eagle's medium (DMEM), Roswell Park Memorial Institute medium (RPMI) 1640, minimum essential medium (MEM), and Iscove's modified Dulbecco's medium (IMDM). In some embodiments, the cell culture medium is a freezing medium comprising dimethyl sulfoxide (DMSO). In some embodiments, the cell culture medium comprises serum, whereas in other embodiments, the cell culture medium is serum free. In some embodiments, the isotonic solution is normal saline. In other embodiments, the isotonic solution is a physiologically acceptable buffer such as phosphate buffered saline. In some embodiments, the medium or solution does not include antibiotics. The engineered monocytes and compositions thereof are provided in some embodiments for use as a medicament.

### IV. Methods for Expressing a Recombinant Protein in a Mammalian Monocyte

Also provided by the present disclosure are methods for expressing a recombinant protein, comprising: a) introducing a nucleic acid comprising a coding region of the recombinant protein into a CD14+ mammalian monocyte to produce a transfected monocyte; and b) culturing the transfected monocyte under conditions to express the recombinant protein, wherein the CD14+ mammalian monocyte is a primary cell or a mortal cultured cell. Primary cells, mortal cultured cells, nucleic acids including expression cassettes and expression vectors for use in the methods are described in Section II above.

In some embodiments, step a) of the methods comprises electroporation of the CD14+ mammalian monocyte. In some embodiments, electroporation of the CD14+ mammalian monocyte comprises exposing the monocyte to: from about 1650 to about 2450 volts for a duration of from 5 to 25 milliseconds for from 1 to 3 energy pulses; from 1650 to 1750 volts for a duration of about 20 milliseconds for 2 energy pulses; about 1700 volts for a duration of about 20 milliseconds for 2 energy pulses; from 1850 to 2450 volts for a duration of from 5 to 25 milliseconds for from 1 to 3 energy pulses; from 2000 to 2300 volts for a duration of from 10 to 20 milliseconds for from 1 to 3 energy pulses; or about 2150 volts for a duration of from about 10 to about 20 milliseconds for 1 or 2 energy pulses. In some preferred embodiments, electroporation of the CD14+ mammalian monocyte is carried out using the Neon® Transfection System (Invitrogen).

In other embodiments, step a) of the methods comprises transduction of the CD14+ mammalian monocyte with a viral vector. In some embodiments, the viral vector is an adeno-associated virus (AAV), such as an AAV serotype 6 (AAV6). In other embodiments, the viral vector is a lentivirus, such as a Baboon endogenous virus glycoprotein-pseudotyped (BaEV) lentivirus.

### ENUMERATED EMBODIMENTS

The following enumerated embodiments are representative of some aspects of the invention.

1. A method for engineering a genome of a CD14+ mammalian monocyte, comprising:

a) introducing a clustered regularly interspaced short palindromic repeats (CRISPR) system into the CD14+ mammalian monocyte to produce a transfected monocyte, wherein the CRISPR system comprises i) at least one guide RNA (gRNA) comprising a sequence that anneals to a target locus of the genome, and ii) an endonuclease or a nucleic acid encoding the endonuclease; and

b) culturing the transfected monocyte to produce an engineered monocyte comprising at least one alteration of the target locus.

2. The method of embodiment 1, wherein the CD14+ mammalian monocyte is a primary cell or a mortal cultured cell.
3. The method of embodiment 1 or 2, wherein the at least one alteration comprises one or more of the group consisting of a disruption of a start codon, a disruption of a splice acceptor sequence, a disruption of a splice donor sequence, and an introduction of a premature stop codon.
4. The method of any one of embodiments 1-3, wherein step a) further comprises introducing a donor nucleic acid into the CD14+ mammalian monocyte, wherein the donor nucleic acid comprises a coding region of a protein of interest flanked on both sides by homology arms to direct insertion of the coding region into the target locus of the genome of the monocyte.
5. The method of embodiment 4, wherein the donor nucleic acid further comprises a promoter in operable combination with the coding region as an expression cassette to direct expression of the protein of interest in the monocyte.
6. The method of embodiment 5, wherein the donor nucleic acid is contained in an expression vector.
7. The method of embodiment 6, wherein the expression vector is a plasmid.
8. The method of any one of embodiments 1-7, further comprising a step before a) of contacting the at least one gRNA with the endonuclease to form a ribonucleoprotein (RNP) complex, and step a) comprises introducing the RNA complex into the CD14+ mammalian monocyte.
9. The method of any one of embodiments 1-8, further comprising a step before a) of isolating the CD14+ mammalian monocyte from peripheral blood mononuclear cells (PBMCs) by positive selection.
10. The method of embodiment 9, wherein the PBMCs were freshly isolated PBMCs obtained from a blood sample or were thawed PBMCs obtained from a cryopreserved aliquot.
11. The method of any one of embodiments 1-11, wherein the target locus is selected from the group consisting of an adeno-associated virus integration site 1 (AAVS1), MAFB, C-MAF, TP53 and PTEN, or wherein the target locus is SIRPα.
12. The method of any one of embodiments 1-11, wherein the endonuclease is a CRISPR-associated protein 9 (Cas9) or a variant thereof.
13. The method of any one of embodiments 1-12, wherein step a) comprises electroporation of the CD14+ mammalian monocyte.
14. The method of embodiment 13, wherein electroporation of the CD14+ mammalian monocyte comprises exposing the monocyte to from about 1650 to about 2450 volts for a duration of from 5 to 25 milliseconds for from 1 to 3 energy pulses, or from 1850 to 2450 volts for a duration of from 5 to 25 milliseconds for from 1 to 3 energy pulses.
15. The method of embodiment 13, wherein electroporation of the CD14+ mammalian monocyte comprises exposing the monocyte to from 1650 to 1750 volts for a duration of about 20 milliseconds for 2 energy pulses, or about 1700 volts for a duration of about 20 milliseconds for 2 energy pulses.
16. The method of embodiment 13, wherein electroporation of the CD14+ mammalian monocyte comprises exposing the monocyte to from 2000 to 2300 volts for a duration of from 10 to 20 milliseconds for from 1 to 3 energy pulses.
17. The method of embodiment 13, wherein electroporation of the CD14+ mammalian monocyte comprises exposing the monocyte to about 2150 volts for a duration of from about 10 to about 20 milliseconds for 1 or 2 energy pulses.
18. The method of any one of embodiments 6-17, wherein the expression vector is a viral vector and step a) comprises transduction of the CD14+ mammalian monocyte with the viral vector after introduction of the CRISPR system.
19. The method of embodiment 18, wherein the viral vector is an adeno-associated virus (AAV), such as an AAV serotype 6 (AAV6) viral vector.
20. The method of embodiment 18, wherein the viral vector is a lentivirus, such as a Baboon endogenous virus glycoprotein-pseudotyped (BaEV) lentiviral vector.
21. The method of any one of embodiments 1-20, wherein the CD14+ mammalian monocyte is a human monocyte.
22. The method of any one of embodiments 1-21, wherein the CD14+ mammalian monocyte is a plurality of cells from which a plurality of transfected monocytes and a plurality of engineered monocytes are produced.
23. The plurality of engineered monocytes of the method of embodiment 22.
24. A composition comprising the plurality of engineered monocytes of embodiment 23 and a cell culture medium or a physiologically acceptable buffer.
25. A method for expressing a recombinant protein, comprising:
a) introducing by electroporation or transduction a nucleic acid comprising a coding region of the recombinant protein into a CD14+ mammalian monocyte to produce a transfected monocyte; and
b) culturing the transfected monocyte under conditions to express the recombinant protein, wherein the CD14+ mammalian monocyte is a primary cell or a mortal cultured cell.
   26. The method of embodiment 25, wherein step a) comprises electroporation of the CD14+ mammalian monocyte.
   27. The method of embodiment 26, wherein electroporation of the CD14+ mammalian monocyte comprises exposing the monocyte to: from about 1650 to about 2450 volts for a duration of from 5 to 25 milliseconds for from 1 to 3 energy pulses; from 1650 to 1750 volts for a duration of about 20 milliseconds for 2 energy pulses; about 1700 volts for a duration of about 20 milliseconds for 2 energy pulses; from 1850 to 2450 volts for a duration of from 5 to 25 milliseconds for from 1 to 3 energy pulses; from 2000 to 2300 volts for a duration of from 10 to 20 milliseconds for from 1 to 3 energy pulses; or about 2150 volts for a duration of from about 10 to about 20 milliseconds for 1 or 2 energy pulses.
   28. The method of embodiment 25, wherein step a) comprises transduction of the CD14+ mammalian monocyte with a viral vector.
   29. The method of embodiment 28, wherein the viral vector is an adeno-associated virus (AAV), such as an AAV serotype 6 (AAV6) viral vector, or a lentivirus, such as a Baboon endogenous virus glycoprotein-pseudotyped (BaEV) lentiviral vector.
   30. The method of any one of embodiments 25-29, wherein the CD14+ mammalian monocyte is a human monocyte.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

In the experimental disclosure which follows, the following abbreviations apply: AAV (adeno-associated virus); CRISPR (clustered regularly interspaced short palindromic repeat); crRNA (CRISPR RNA); DC (dendritic cell); DSB (double strand break); eGFP (enhanced GFP); GFP (green fluorescent protein); GOI (gene of interest); gRNA (guide RNA); HA (homology arm); HDR (homology directed repair); indels (insertions and deletions); MOI (multiplicity of infection); ms (milliseconds); NHEJ (non-homologous end joining); PAM (protospacer adjacent motif); PBMC (peripheral blood mononuclear cells); TIDE (Tracking of Indels by DEcomposition); and tracrRNA (trans-activating crRNA).

### Example 1: General Materials and Methods

Reagents

Incomplete medium: 500 mL RPMI (+L-glutamine) supplemented with 10% heat-inactivated Fetal Bovine Serum (HI-FBS), and without penicillin/streptomycin. This medium was used for resting monocytes after isolation or monocyte cryopreservation.
Complete medium: 500 mL RPMI (+L-glutamine) supplemented with 10% heat-inactivated Fetal Bovine Serum (HI-FBS), 20 ng/mL M-CSF, 20 ng/mL IL-3, 20 ng/mL IL-34, 100 U/mL Penicillin, and 100 ug/mL Streptomycin.
Freezing medium: CryoStor CS10.
Cell separation reagents: Human Monocyte Isolation Kit (Stem Cell Technologies).
Electroporation reagents: Neon 10 uL Transfection Kit (Invitrogen).

Isolation of peripheral blood mononuclear cells (PBMCs): Peripheral blood was transferred into a 50 mL conical tube. 10 mL ACK (ammonium-chloride-potassium) lysis buffer was added to the conical tube, and the solution was incubated at room temperature for 5 minutes. The volume of the solution was adjusted to 50 mL using PBS, after which it was centrifuged at 1,800 rpm for 5 minutes with no brake. The lysed blood (supernatant) was removed, and the cell pellet was washed with 35 mL 1×PBS and centrifuged at 1,500 rpm for 5 minutes. The steps of adding the ACK lysis buffer, centrifugation, and washing the cell pellet were repeated until the cell pellet appeared white. Cells were then either frozen or used immediately and purified.

Isolation of CD14⁺ monocytes: The density of the isolated PBMCs was adjusted to 5×10⁷ cells/mL before the cells were transferred to a 14 mL polystyrene round-bottom tube. Monocytes were isolated using the Human Monocyte Isolation Kit (Stem Cell Technologies) according to the manufacturer's protocol. The isolated monocytes were counted, analyzed for purity using flow cytometry (based on the percentage of CD14⁺ cells), and re-suspended to a density of 1×10⁷ cells/mL in CryoStor CS10, and cryopreserved at −80° C.

Flow cytometry: Cells were washed with chilled PBS and stained with anti-human CD14 antibody. Analysis was performed using a flow cytometer (BD Biosciences) and the FlowJo software (Treestar).

Monocyte culture: Monocytes were cultured in monocyte complete medium at a density of 5×10⁵ cells/mL at 37° C. and 5% CO2 level. Fresh medium supplemented with fresh cytokines was added every 3 days, and the medium was completely replaced after 6 days of culturing.

### Example 2: GFP Expression in Monocytes by Electroporation

### Materials and Methods

Monocytes were rested in the incomplete media without antibiotics for at least an hour. The cells were washed in PBS and then re-suspended in T Buffer (Invitrogen) to a density of 3-5×10⁸ cells/mL. 1 μg of eGFP mRNA (Trilink) was added to the cells prior to electroporation using the Neon Transfection system. Cells were electroporated using 1-2 pulses of 2100-2150 volts, for 10-20 milliseconds. Additionally, cells were electroporated using 3 pulses of 1400 volts for about 10 millisecond or 2 pulses of about 1700 volts for about 20 milliseconds. Following electroporation, cells were plated in the monocyte complete media with the media being refreshed every 3 days.

### Results

To optimize the electroporation protocol for transfecting primary human monocytes, several conditions were tested for delivering eGFP-encoding mRNAs into primary human monocytes. About 24 hours after electroporation of primary human monocytes using the Neon Transfection Kit (Invitrogen), the percentage of GFP-positive monocytes was measured using flow cytometry. T lymphocytes can be efficiently transfected by an electroporation condition involving 3 pulses at 1400 volts for 20 milliseconds (ms). Three different electroporation conditions, including two pulses at 2150 volts for 10 ms each, one pulse at 2150 volts for 10 ms, and one pulse at 2150 volts for 20 ms were tested along with a no electroporation control, and the T lymphocyte electroporation condition. Additionally, one pulse at 1700 volts for 20 ms was tested. The GFP-positive monocytes were further analyzed for viability using the e-Fluor 780 Fixable Viability Dye. Table 2-1 depicts the percentages of GFP-positive and viable monocytes obtained under the respective conditions. Surprisingly, the condition suitable for transfection of T lymphocytes was found to be inefficient for transfection of monocytes, resulting in a modest percentage (31.8%) of GFP-positive monocytes. In contrast, electroporation using 2 pulses at 2150 volts for 10 ms resulted in a high percentage of GFP-positive (91.6%) and viable (87.3%) monocytes. However, electroporation using 2 pulses at 1700 volts for 20 ms resulted in the highest percentage of GFP-positive (94.3%) and viable (97.9%) monocytes as compared to the other protocols.

TABLE 2-1
Electroporation Efficiency

| Electroporation condition | % GFP-positive monocytes | % viability |
|---|---|---|
| No electroporation | 0.351 | 87.3 |
| 1400 volts, 10 ms, 3 pulses | 31.8 | 97.9 |
| 1700 volts, 20 ms, 2 pulses | 94.3 | 97.9 |
| 2150 volts, 10 ms, 1 pulse | 87.9 | 81.1 |
| 2150 volts, 10 ms, 2 pulses | 91.6 | 87.3 |
| 2150 volts, 20 ms, 1 pulse | 89.6 | 81.6 |

### Example 3: Gene Editing in Monocytes Using CRISPR/Cas9

### Materials and Methods

Monocyte preparation: Cryopreserved monocytes were thawed in a 37° C. water bath, and rested in monocyte incomplete medium in an untreated culture plate for 4 hours at 37° C., at 5% CO2 level with humidity. The monocytes were then collected into 50 mL conical tubes and centrifuged at 400 g for 5 minutes. The monocytes were washed with PBS and then resuspended in T Buffer (Invitrogen) to a final density of 5×10⁷ cells/mL.

CRISPR/Cas9 reagent preparation: The crRNA:tracrRNA (gRNA) duplex was prepared by mixing 200 μM of Alt-R® CRISPR-Cas9 crRNA and 200 μM of Alt-R® CRISPR-Cas9 tracrRNA (Integrated DNA Technologies, Inc., Skokie, Ill.) in nuclease-free IDTW buffer, heating the mixture at 95° C. for 5 minutes, and then cooling to room temperature. The RNP complexes were prepared by mixing 1 μg of gRNA duplex (e.g., gRNA for MAFB, c-MAF, TP53, PTEN, or SIRPα) with 3 μg of Cas9 protein according to the manufacturer's protocol, followed by incubation for 20 minutes at room temperature before use. The crRNAs and tracrRNAs included chemical modifications to increase nuclease resistance and reduce innate immune responses (e.g., 2′-O-methyl, 2′-O-methyl-3′-phosphorothioate, 2′-O-methyl-3′-thiophosphonoacetate, etc.). The locus-specific gRNA sequences used are listed in Table 3-1.

TABLE 3-1
Guide RNA Sequence Specificity\hat{ }

| Target Name | Target Sequence (SEQ ID) | PAM |
|---|---|---|
| MAFB | CTACCAGCAGATGAACCCCG (NO: 1) | AGG |
| c-MAF1 | CGACCTGCCCACCAGTCCCC (NO: 2) | TGG |
| TP53 | CCCCTTGCCGTCCCAAGCAA (NO: 3) | TGG |
| PTEN | GCTAACGATCTCTTTGATGA (NO: 4) | TGG |
| SIRPα | CTGAAACAGTTGTTACCCGG (NO: 5) | GGG |

Alt-R ® CRISPR-Cas9 crRNA sequences include an additional 16-22 nucleotides to facilitate annealing to Alt-R ® CRISPR-Cas9 tracrRNA.

Neon Electroporation: The RNP complex was added to the monocytes prior to electroporation using the Neon Transfection Kit (Invitrogen). 1 μg of GFP mRNA (Trilink) was added to the cells as a transfection efficiency reporter. Using the 10 μL electroporation tips, 10 μL of the cell mixture was electroporated using 2 pulses at 2150 volts for 10 milliseconds. Following electroporation, cells were transferred to 0.5 mL of the monocyte complete medium (without penicillin/streptomycin) and rested for one hour at 37° C. under 5% CO2 and with humidity. 0.5 mL of the monocyte complete medium with 2× penicillin/streptomycin was then added to the cell culture, and the cells were incubated for another 5 days. The medium was changed on Day 3. On Day 5, cells were analyzed for editing efficiency using flow cytometry.

Tracking of Indels by DEcomposition (TIDE): Analysis of genome alternation was carried out as described (Brinkman et al., Nucleic Acids Research 42(22):e168, 2014) to determine the editing efficiency and the predominant types of insertions and deletions (indels) in the CRISPR/Cas9-edited DNA sequences. Briefly, genomic DNA was extracted from monocytes 5 days after transfection, and a 400-1500 bp region around the editing site was PCR amplified from the genomic DNA and subjected to sequencing and analysis.

TABLE 3-2
Primer Sequences

| Target Name | Forward Primer | Reverse Primer |
|---|---|---|
| MAFB | TCAACGACTTCGACCTGCTC | GTGATGGTGGTGGTGGTGAG |
| | (SEQ ID NO: 6) | (SEQ ID NO: 7) |
| c-MAF | GAGCGAGGGAGCACATTGG | GCGCACCTGGAAGACTACTA |
| | (SEQ ID NO: 8) | (SEQ ID NO: 9) |
| TP53 | TGCTCTTGTCTTTCAGACTTCC | GGAAGGGACAGAAGATGACAGG |
| | (SEQ ID NO: 10) | (SEQ ID NO: 11) |
| PTEN | CCAGGCCTCTGGCTGCTGAG | CGGACAATAGCCCTCAGGAAG |
| | (SEQ ID NO: 12) | (SEQ ID NO: 13) |
| SIRPα | TGCAGGTTTGTTGTGAGGGT | GCTCCCTTTCCGGAACTTCA |
| | (SEQ ID NO: 14) | (SEQ ID NO: 15) |

### Results

To establish the feasibility of genome editing in monocytes, the Alt-R® CRISPR/Cas9 System (Integrated DNA Technologies), was used to target the MAFB, c-MAF, TP53, PTEN, and SIRPα genes in monocytes. TIDE analysis was conducted to determine the editing efficiency and indel spectra of the five target genes. 87.2% of the cMAF1 sequences in the CRISPR/Cas9-edited cell pool carried an indel, with 70.3% being a −1 deletion, 3.2% being a −2 deletion, 7.7% being a −3 deletion and 4.4% being a −6 deletion. 91.4% of the MAFB sequences in the CRISPR/Cas9-edited cell pool carried an indel, with 49.1% being a −1 deletion, 32.5% being a −2 deletion, 2.8% being a −3 deletion, and 3.7% being a +2 insertion. 56% of the TP53 sequences in the CRISPR/Cas9-edited cell pool carried an indel, with 43.1% being a +1 insertion, 7.6% being a −1 deletion, and 3.8% being a −3 deletion. 62.9% of the PTEN sequences in the CRISPR/Cas9-edited cell pool carried an indel, with 60.7% being a −1 deletion and 2.2% being a −3 deletion. 69.9.% of the SIRPα sequences in the CRISPR/Cas9-edited cell pool carried an indel. Overall editing efficiency is shown in Table 3-3. These results demonstrate that monocyte genome can be efficiently edited using the CRISPR/Cas9 system.

TABLE 3-3
Editing Efficiency

| Gene Name | overall editing efficiency (%) |
|---|---|
| cMAF1 | 87.2 |
| MAFB | 91.4 |
| P53 | 56.0 |
| PTEN | 62.9 |
| SIRPa | 69.9 |

### Example 4: GFP Expression in Monocytes by Lentivirus Transduction

### Materials and Methods

Lentivirus production: HEK293T cells suspended in DMEM containing 10% Fetal Bovine Serum (FBS) were seeded on a T150 flask coated with 0.1% gelatin a night prior to transfection to achieve 50-70% confluency. Transfection reagents were prepared by mixing 10 μg of plasmid expressing Baboon envelope (pBaEV), 20 μg of a plasmid expressing GAG and POL (psPAX2) and 30 μg of a pLL or pRRL plasmid expressing GFP under regulation of MND promoter into Gibco® Opti-MEM™ medium (Thermo Fisher), followed by incubation for 5 minutes at room temperature. The pBaEV plasmid was obtained from colleagues (Fusil et al., Molecular Therapy, 23:1734-47, 2015), and the psPAX2, pLL and pRRL plasmids were obtained from Addgene. The plasmid mixture was then placed into Gibco® Opti-MEM™ medium containing Lipofectamine® 2000 (Invitrogen) and incubated at room temperature for 30 minutes. The mixture was then transferred into the T150 flask containing HEK293T cells and incubated at 37° C. under 5% CO2 and humidity for 6 hours. The transfection medium was removed and replenished with DMEM containing 20% FBS. The first viral harvest was collected at 24 hours post transfection and then replenished with fresh DMEM containing 20% FBS. The second viral harvest was collected at 48 hours post transfection. Viral titers were measured by RT-qPCR.

Transduction: Cryopreserved monocytes were thawed and rested in the incomplete medium for 4 hours. The rested monocytes were counted and plated in a 24-well non-treated culture plate with monocyte complete medium at 1×10⁶ cells/mL. Integration-deficient Baboon-pseudotyped lentivirus expressing GFP was added to the cells at an MOI of 20. The plate was centrifuged at 700×g for 1 hour at room temperature, and incubated at 37° C. under 5% CO2 and humidity for 5 hours. The medium was replaced with 1 mL of fresh monocyte complete medium, and after which the cells were further incubated for 3 days. Following incubation, the cells were collected and GFP-positive cells were analyzed using flow cytometry.

### Results

It has been reported that VSV-G pseudotyped lentivirus was unable to transduce monocytes (Muhlebach et al., Molecular Therapy, 12:1206-1215, 2005). To determine whether lentivirus with alternative pseudotypes are suitable for the transduction of monocytes, BaEV-pseudotyped lentivirus expressing eGFP (Fusil et al., Molecular Therapy, 23:1734-47, 2015) was used to transduce primary human monocytes. Monocytes that were not treated with lentivirus were used as controls. Cells were analyzed using flow cytometry 5 days after lentiviral transduction or control treatment. Table 4-1 shows the percentages of GFP-expressing primary human monocytes 5 days after transduction with eGFP-expressing lentivirus Lenti-pLL, eGFP-expressing lentivirus Lenti-pRRL, or no lentivirus control. Gating of live cells was performed based on the incorporation of the eFluor780 Fixable Viability Dye. Close to 25% of the monocytes were GFP-positive upon Lenti-pRRL transduction. These results demonstrate that monocytes can be transduced using the BaEV-pseudotyped lentivirus.

TABLE 4-1
Transduction Efficiency

| Condition | % GFP-positive monocytes |
|---|---|
| No Lentiviral transduction | 0.761 |
| pLL Lentiviral transduction | 2.85 |
| pRRL Lentiviral transduction | 24.8 |

### Example 5: GFP Expression in Monocytes by AAV Transduction

Previously, monocytes were found to be susceptible to infection with various AAV vectors (Grimm et al., J Virol, 82:5887-5911, 20018). To determine whether monocytes could be engineered to stably express a gene of interest via AAV-mediated homology-directed repair (HDR), a promoter-less splice acceptor AAV6 vector encoding the eGFP reporter gene (Vigene Biosciences) and flanked by homology arms to a CRISPR-targeted site (e.g., AAVS1) was used as a donor template upon induction of a double strand break at the target site. Briefly, rested monocytes were electroporated as previously described with 2 pulses at 1700 volts for 20 milliseconds each in the presence of an RNP complex formed from an AAVS1 gRNA and Cas9 protein. Monocytes received the AAVS1 gRNA without Cas9 protein as a negative control. Monocytes were rested for 30 minutes post-electroporation in monocyte complete medium (without penicillin/streptomycin) at 37° C. under 5% CO2 and with humidity before addition of an equal volume of monocyte complete medium with 2× penicillin/streptomycin. Immediately after electroporation, the electroporated monocytes were transduced with AAV serotype 6 at an MOI of 5×10⁵ vg/cell. Upon successful HDR, eGFP was expressed under the promoter of the target gene (e.g., AAVS1 promoter). CRISPR reagents were introduced using the Neon Transfection Kit (Invitrogen) as described above. Five days after transduction, the engineered monocytes were subjected to flow cytometry analysis to determine the percentage of eGFP-positive cells, which is indicative of the efficiency of HDR.

### Results

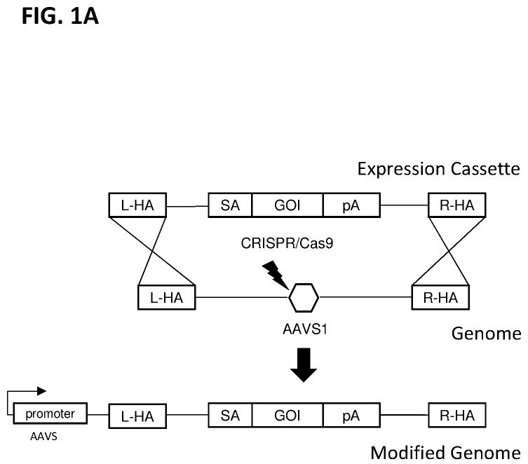

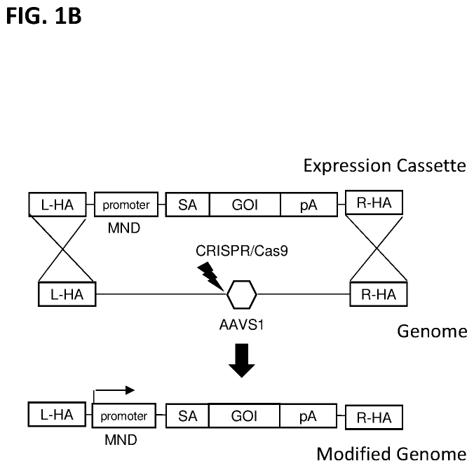

An AAV6 vector was found to be effective in delivering a donor DNA template (eGFP) to primary monocytes for homology directed repair of a CRISPR/Cas9-mediated double-stranded break. Specifically, the combination of a CRISPR/Cas9 system and an AAV6 vector as illustrated in FIG. 1A was found to mediate integration of a eGFP into the monocyte genome resulting in 7.79% GFP-positive monocytes, while no GFP-positive monocytes was detected in the control sample (Table 5-1).

TABLE 5-1
Efficiency of Expression of Gene of Interest

| | % GFP-positive |
|---|---|
| Condition | monocytes |
| AAV SA-GFP only (control) + AAV6 | 0.14 |
| CRISPR/Cas9 and AAV SA-GFP + AAV6 | 7.73 |

We claim:

1. A method for engineering a genome of a CD14+ mammalian monocyte, comprising:
a) introducing a clustered regularly interspaced short palindromic repeats (CRISPR) system into the CD14+ mammalian monocyte to produce a transfected monocyte, wherein the CRISPR system comprises i) at least one guide RNA (gRNA) comprising a sequence that anneals to a target locus of the genome, and ii) an endonuclease or a nucleic acid encoding the endonuclease; and
b) culturing the transfected monocyte to produce an engineered monocyte comprising at least one alteration of the target locus.

2. The method of claim 1, wherein the CD14+ mammalian monocyte is a primary cell or a mortal cultured cell.

3. The method of claim 1, wherein the at least one alteration comprises one or more of the group consisting of a disruption of a start codon, a disruption of a splice acceptor sequence, a disruption of a splice donor sequence, and an introduction of a premature stop codon.

4. The method of claim 1, wherein step a) further comprises introducing a donor nucleic acid into the CD14+ mammalian monocyte, wherein the donor nucleic acid comprises a coding region of a protein of interest flanked on both sides by homology arms to direct insertion of the coding region into the target locus of the genome of the monocyte.

5. The method of claim 4, wherein the donor nucleic acid further comprises a promoter in operable combination with the coding region as an expression cassette to direct expression of the protein of interest in the monocyte.

6. The method of claim 5, wherein the donor nucleic acid is contained in an expression vector.

7. The method of claim 6, wherein the expression vector is a plasmid.

8. The method of claim 1, further comprising a step before a) of contacting the at least one gRNA with the endonuclease to form a ribonucleoprotein (RNP) complex, and step a) comprises introducing the RNA complex into the CD14+ mammalian monocyte.

9. The method of claim 1, further comprising a step before a) of isolating the CD14+ mammalian monocyte from peripheral blood mononuclear cells (PBMCs) by positive selection.

10. The method of claim 9, wherein the PBMCs were freshly isolated PBMCs obtained from a blood sample or were thawed PBMCs obtained from a cryopreserved aliquot.

11. The method of claim 1, wherein the target locus is selected from the group consisting of an adeno-associated virus integration site 1 (AAVS1), MAFB, C-MAF, TP53, PTEN, and SIRPα.

12. The method of claim 1, wherein the endonuclease is a CRISPR-associated protein 9 (Cas9) or a variant thereof.

13. The method of claim 1, wherein step a) comprises electroporation of the CD14+ mammalian monocyte.

14. The method of claim 13, wherein electroporation of the CD14+ mammalian monocyte comprises exposing the monocyte to from about 1650 to about 2450 volts for a duration of from 5 to 25 milliseconds for from 1 to 3 energy pulses, or from 1850 to 2450 volts for a duration of from 5 to 25 milliseconds for from 1 to 3 energy pulses.

15. The method of claim 13, wherein electroporation of the CD14+ mammalian monocyte comprises exposing the monocyte to from 1650 to 1750 volts for a duration of about 20 milliseconds for 2 energy pulses, or about 1700 volts for a duration of about 20 milliseconds for 2 energy pulses.

16. The method of claim 13, wherein electroporation of the CD14+ mammalian monocyte comprises exposing the monocyte to from 2000 to 2300 volts for a duration of from 10 to 20 milliseconds for from 1 to 3 energy pulses.

17. The method of claim 13, wherein electroporation of the CD14+ mammalian monocyte comprises exposing the monocyte to about 1950 volts for a duration of from about 10 to about 20 milliseconds for 1 or 2 energy pulses.

18. The method of claim 6, wherein the expression vector is a viral vector and step a) comprises transduction of the CD14+ mammalian monocyte with the viral vector after introduction of the CRISPR system.

19. The method of claim 18, wherein the viral vector is an adeno-associated virus serotype 6 (AAV6) viral vector.

20. The method of claim 18, wherein the viral vector is a Baboon endogenous virus glycoprotein-pseudotyped (BaEV) lentiviral vector.

21. The method of claim 1, wherein the CD14+ mammalian monocyte is a human monocyte.

22. The method of claim 1, wherein the CD14+ mammalian monocyte is a plurality of cells from which a plurality of transfected monocytes and a plurality of engineered monocytes are produced.

23. The plurality of engineered monocytes of the method of claim 22.

24. A composition comprising the plurality of engineered monocytes of claim 23 and a cell culture medium or a physiologically acceptable buffer.

25. A method for expressing a recombinant protein, comprising:
a) introducing by electroporation or transduction a nucleic acid comprising a coding region of the recombinant protein into a CD14+ mammalian monocyte to produce a transfected monocyte; and
b) culturing the transfected monocyte under conditions to express the recombinant protein, wherein the CD14+ mammalian monocyte is a primary cell or a mortal cultured cell.

26. The method of claim 25, wherein step a) comprises electroporation of the CD14+ mammalian monocyte.

27. The method of claim 26, wherein electroporation of the CD14+ mammalian monocyte comprises exposing the monocyte to: from about 1650 to about 2450 volts for a duration of from 5 to 25 milliseconds for from 1 to 3 energy pulses; from 1650 to 1750 volts for a duration of about 20 milliseconds for 2 energy pulses; about 1700 volts for a duration of about 20 milliseconds for 2 energy pulses; from 1850 to 2450 volts for a duration of from 5 to 25 milliseconds for from 1 to 3 energy pulses; from 2000 to 2300 volts for a duration of from 10 to 20 milliseconds for from 1 to 3 energy pulses; or about 2150 volts for a duration of from about 10 to about 20 milliseconds for 1 or 2 energy pulses.

28. The method of claim 25, wherein step a) comprises transduction of the CD14+ mammalian monocyte with a viral vector.

29. The method of claim 28, wherein the viral vector is an adeno-associated virus serotype 6 (AAV6) viral vector, or a Baboon endogenous virus glycoprotein-pseudotyped (BaEV) lentiviral vector.

30. The method of claim 25, wherein the CD14+ mammalian monocyte is a human monocyte.
